(19) United States
(12) Patent Application Publication
(10) Pub. No.: US 2020/0154860 A1
(43) Pub. Date: May 21, 2020

(54) Automatic Home Use Machine for Mixing and Preparing Custom-Fit Personal Care Products

(71) Applicant: Po Sheung Denny Lee, Hong Kong (CN)

(72) Inventor: Po Sheung Denny Lee, Hong Kong (CN)

(21) Appl. No.: 16/191,988

(22) Filed: Nov. 15, 2018

(51) Int. Cl. A45D 44/00; A45D 34/00

(52) U.S. Cl. CPC A45D 44/00; A61K 8/02; A45D 34/00

(57) ABSTRACT

An apparatus for preparing customized skin care products (hereafter, “the apparatus”) including a first, second, and third portion and a control unit working together to blend a liquid and an additive used to formulate the skin care product. The first portion includes a hotplate for heating the liquid and the additive, a stirrer for mixing the liquid and the additive, a thermometer, and a scale. The second portion includes a container, which the liquid and additive are received in to be heated and mixed. The third portion includes a dispenser for releasing the additive into the liquid within the container. To administer the formulation process, the control unit receives inputs internal and external to the apparatus and outputs signals to the first, second, and/or third portions. When the control unit outputs the signals, the hotplate, stirrer, thermometer, scale, and/or dispenser are activated to prepare the skin care product.

### BACKGROUND

The present disclosure relates to preparation of personal care products. More particularly, the present disclosure relates to an automatic machine for mixing and/or preparing personal care products.

The condition of a person's skin, hair, and/or body may be degraded through exposure to environmental factors, either naturally occurring (sunlight, wind abrasion, humidity, etc.) or man-made (heating, air condition, pollutants, etc.), pathological processes such as dermatological diseases, or the normal aging process. The various elements to which the skin, hair, and/or body are exposed may act individually or synergistically.

To correct or prevent the deterioration of the condition of a person's skin, hair, and/or body that may occur over time, consumers have increasingly sought new and improved cosmetic, dermatological, and vitamin compositions for personal care. Many prior art personal care products that are currently available are provided to consumers as pre-prepared, mass-produced formulations. The prior art pre-prepared personal care products have limited transparency regarding their ingredients. Additionally, a user is not able to alter the formulation of the pre-prepared prior art personal care products to accommodate her or his unique skin, hair, and/or body characteristics and personal preferences for specialized treatment. The user is also uncertain of the age and freshness of the pre-prepared prior art formulations, which may have been prepared long before the product was sold to the user. Further, the user bears the burden of paying marked-up costs (manufacturing, marketing, unnecessary ingredients and amount of product, etc.) for the pre-prepared personal care products.

There is a need for a home use machine that can prepare personalized, cost-efficient personal care products for an individual user which are customized and tailored to the needs and preferences of the user.

### SUMMARY

The disclosed apparatus for preparing customized personal care products (hereafter, “the apparatus”) is configured for individual use in the home of a user. The apparatus may be used with an application on a mobile device having a camera to analyze a condition of an area on a user's body to determine a formulation for the custom personal care product.

The apparatus includes a first, second, and third portion and a control unit which work together to blend a liquid and an additive used to formulate the custom skin, hair, and/or body care products. Though the apparatus is contemplated to prepare all kinds of personal care products, the apparatus will be discussed hereafter in the context of a skin care product. The first portion of the apparatus includes a hotplate for heating the liquid and the additive, a stirrer for mixing the liquid and the additive, a thermometer for determining the temperature of the liquid and the additive, and a scale for determining the weight of the liquid and the additive. The second portion includes a container defining a chamber, which the liquid and additive are received in to be heated and mixed. The third portion includes a dispenser defining a plurality of cavities for releasing the additive into the liquid within the chamber.

To begin and administer the formulation process, the control unit receives an input internal and external to the apparatus and outputs signals to the first, second, and/or third portions. When the control unit outputs the signals, the hotplate, stirrer, thermometer, scale, and/or dispenser are activated to prepare the customized skin care product.

The additive may come in the form of a pill which is housed in a pill cartridge. The pill cartridge has a unique electronic label which is identified by an electronic label reader employed by the apparatus. The additive is recommended and/or selected based upon the condition of the user's skin as determined by the analysis done by the application. After the analysis, the recommended and/or selected pill cartridge is sent to the user for use in the apparatus.

### BRIEF DESCRIPTION OF THE DRAWINGS

Aspects of a disclosed embodiment will be described in reference to the drawings, where like numerals reflect like elements:

FIG. 1 is a front exploded view, partly in schematic, of an apparatus for preparing customized skin care products (hereafter, “the apparatus”) according to aspects of the disclosure;

FIG. 2 is a front view, partly in schematic, of a lower portion of the apparatus of FIG. 1;

FIG. 3A is a front view, partly in schematic, of an additive cartridge of the apparatus of FIG. 1;

FIG. 3B is a front view, partly in idealized form, of an upper portion of the apparatus of FIG. 1, and

FIG. 4 is a top view, partly in schematic, of a control unit of the apparatus of FIG. 1.

### DETAILED DESCRIPTION

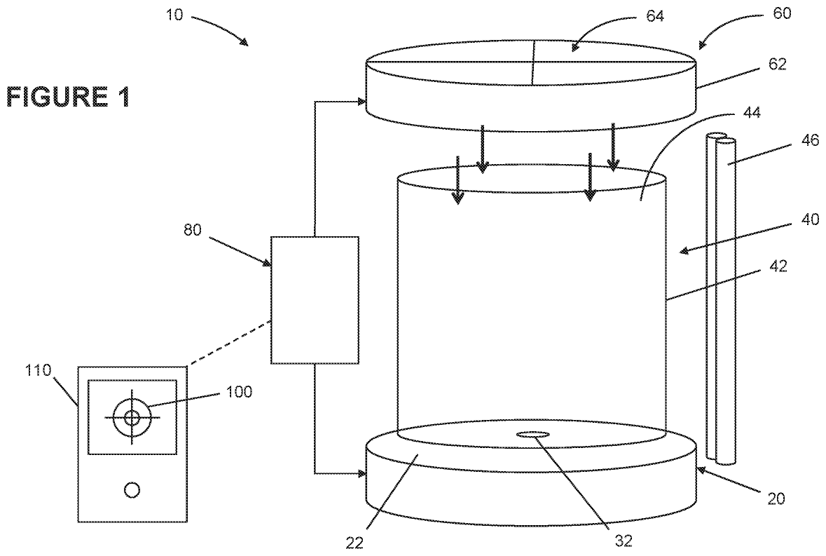

An embodiment of an apparatus and a method for preparing customized skin care products (hereafter, “the apparatus”) according to aspects of the disclosure will now be described with reference to FIGS. 1-4. The apparatus will generally be referred to by the reference numeral 10. Various materials, methods of preparation, methods of construction, and methods of manufacture will be discussed in the context of the disclosed embodiment. Those skilled in the art will recognize known substitutes for the materials, preparation methods, construction methods, and manufacturing methods, all of which are contemplated as compatible with the disclosed embodiment and are intended to be encompassed by the appended claims.

The apparatus 10 is comprised of features configured to analyze a portion of skin on a body of a user to provide feedback relating to the condition of the skin of the user, recommend a customized skin care formulation for a skin care product based upon the condition of the skin of the user, and prepare/formulate the skin care product based upon the condition of the skin of the user and/or based upon a selection made by the user. The apparatus 10 is also capable of analyzing user feedback, formulations, and user behavior to increase effectiveness of the recommendations and skin care products in order to improve the condition of the skin of the user.

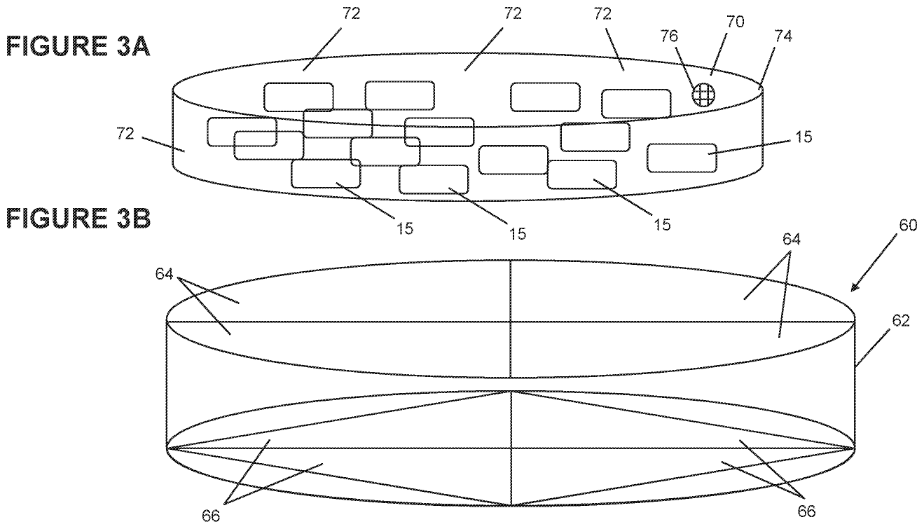

As shown in FIG. 1 of the disclosed embodiment, the apparatus 10 is configured to select and combine one or more liquid with one or more additive 15. In the disclosed embodiment, the liquid is purified water but those having skill in the art will realize that other liquids may be used for formulating a skin care product. The additive 15, shown in FIG. 3A, in the disclosed embodiment is any ingredient that can combine with the liquid and/or another ingredient to formulate a skin care product. In the disclosed embodiment, the additive 15 is in the form of a pill.

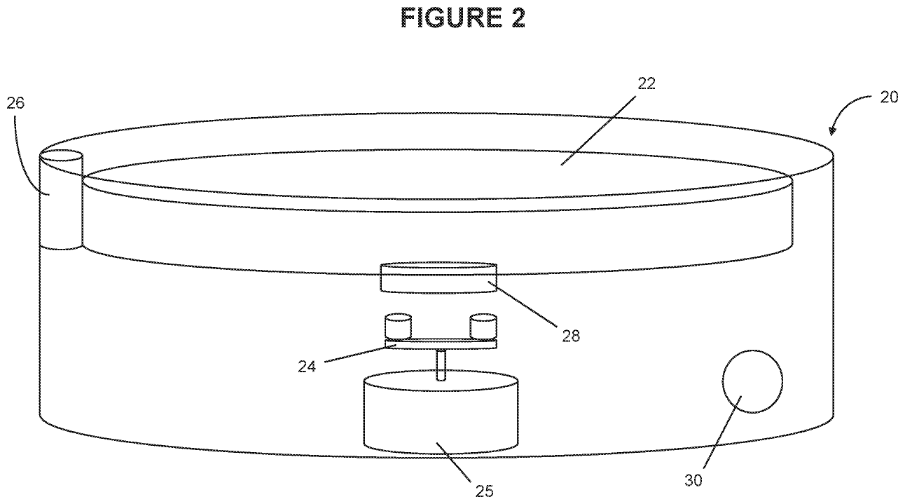

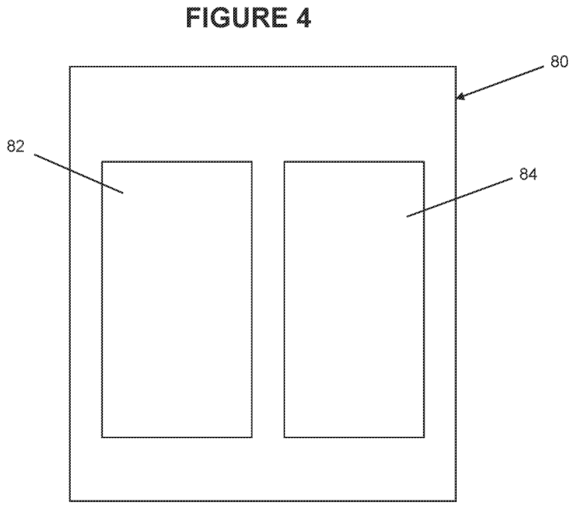

The apparatus 10 comprises a lower (first) portion 20 (see FIG. 2), a middle (second) portion 40 (see FIG. 1), an upper (third) portion 60 (see FIGS. 3A and 3B), and a control unit 80 (see FIG. 4). The lower portion 20 includes a hotplate 22 for heating the liquid and/or the additive 15, a stirrer 24 for stirring/mixing the liquid and the additive 15, a thermometer 26 for determining the temperature of the liquid and/or the additive 15, and a scale 28 for determining the weight and/or volume of the liquid and/or the additive 15. The middle portion 40 includes a container 42 defining a chamber 44 for receiving the liquid and the additive 15. The upper portion 60 includes a dispenser 62 defining a plurality of cavities 64 for releasing the additive 15 into the chamber 44. The control unit 80 receives inputs both internal to and external of the apparatus 10 and outputs a signal to one or more of the components included by the first, second, and third portions 20, 40, 60. In this arrangement, the first portion 20, second portion 40, third portion 60, and control unit 80 cooperate with each other to prepare the customized skin care product.

Referring to FIG. 1 of the disclosed embodiment, the container 42 is constructed of a cylindrical glass jar arranged between the dispenser 62 of the upper portion 60 and the hot plate 22 of the lower portion 20. The container 42 is configured to be removable so that the skin care product can be poured into a separate receptacle for usage once it is finished.

Referring to FIG. 2 of the disclosed embodiment, the lower portion 20 is comprised of the hotplate 22, the stirrer 24, the thermometer 26, and the scale 28. The lower portion 20 may also include the start button 30 to begin the process of formulating the skin care product. In the disclosed embodiment, the hotplate 22 is disk-shaped and positioned below the container 44 so that heat is applied directly to contents of the container 44. The stirrer 24 of the disclosed embodiment is a magnetic stirrer 24 which employs a motor 25 having a rotating magnetic field or stationary electromagnets which cause a stir bar 32 placed within the container to spin quickly, stirring the liquid and the additive 15. The thermometer 26 of the disclosed embodiment is a digital thermometer 26 and is positioned adjacent the hotplate 22 for determining the temperature of the liquid prior to dispensing the additive 15 into the container 44 and/or determining the temperature of the liquid combined with the additive 15 in the container 42. The scale 28 of the disclosed embodiment is an electronic scale 28 configured to determine the weight and/or volume of the liquid prior to dispensing the additive 15 into the container 42 or the weight and/or volume of the liquid combined with the additive 15 in the container 42.

As shown in FIG. 3B of the disclosed embodiment, the dispenser 62 is positioned above the container 42 so the additive 15 can be released into the container 42 and stirred with the liquid to formulate the skin care product. In the disclosed embodiment, the dispenser 62 defines four cavities 64 for receiving four different groups of additives 15. Each cavity 64 includes a gate 66 configured to open, independently of each other, to dispense the additive 15 into the container 42. Each gate 66 is controlled by an actuator (not shown) wired to a microprocessor 82 (see FIG. 4) included in the control unit 80.

Referring to FIG. 3A of the disclosed embodiment, the additive 15 is comprised of one or more different groups of additives 15 which are each composed of one or more different ingredients. Each group of additives 15 is stored in a cartridge 70 in the dispenser 62 prior to being released into the cavity 64 to be dispensed into the container 42. The cartridge 70 defines a plurality of receptacles 72 which are sealed by an aluminum foil cover 74. Each receptacle 72 corresponds with a cavity 64 defined by the dispenser 62. The cartridge cover 74 is arranged in facing contact with the cavities 64 of the dispenser 62 so that when a group of additives 15 is selected, the cover 74 of the cartridge 70 is punctured and broken to allow the additive 15 to release into a corresponding cavity 64.

The control unit 80 includes a microprocessor 82 that is connected and/or wired to the start button 30, an RFID reader 84 within the control unit 80, the gates 66, the hotplate 22, the motor 25 of the stirrer 24, the scale 28, the thermometer 26, and a UV-C light 46 used to sterilize the apparatus 10. The UV-C light 46 sterilizes an interior of the apparatus 10 by killing bacteria and/or viruses which may be present within the apparatus 10. Due to the wired connections, the control unit 80 is capable of activating or deactivating the components of the apparatus 10.

The apparatus 10 is configured to communicate and cooperate with a program and/or application 100 employed by a mobile device 110 having a camera. In the disclosed embodiment, the mobile device 110 is a cell phone of the user, but one having skill in the art will recognize that other mobile devices such as a tablet, laptop, desktop computer, and/or camera, may be compatible with the apparatus 10. The application 100 is used by the user to capture images of the skin of the user and analyze the images to determine the condition of the skin of the user. The analysis of the images is then used to provide feedback and make recommendations for the selection of additives 15 and/or skin care products based upon the analysis of the condition of the skin of the user. The user can then select additives 15 for purchase through the application 100. The cartridge 70 storing the additive 15 is then sent to the user for use within the apparatus 10. The cartridge 70 storing a selected additive 15 includes a distinct RFID label 76 which can be read by the RFID reader 84 of the apparatus 10. The application 100 may also employ artificial intelligence to study consumer feedback, formulations of skin care products, and additive 15 selection to increase the efficiency of the application 100.

When a new cartridge 70 is added to the dispenser 62 of the disclosed embodiment, the RFID reader 84 reads the RFID label 76 on the cartridge 70 which sends information regarding the identification of the additive 15 within the cartridge 70 to the microprocessor 82. Based on the identification (RFID) of the additive 15, the formulation process for the skin care product is programmed into the microprocessor 82, which enables the microprocessor 82 to send appropriate signals to the gates 66, the hotplate 22, the stirrer 24, the scale 28, the thermometer 26, and the UV-C light 46 in order to formulate the skin care product.

In the disclosed embodiment, a specified volume of purified water is poured into the container 42 for formulation. Once the container 42 is filled with the specified amount of water, the container 42 is placed back into the apparatus 10 and a door (not shown) on the apparatus is closed and locked. The door is wired to the microprocessor 82 so that pushing the start button 30 on the lower portion 20 of the apparatus 10 enables the microprocessor 82 to ensure that the door on the apparatus 10 is locked. The scale 28 then weighs the water to determine that the volume of the water within the container 42 is the specified amount of water needed for the chosen skin care product formulation. If the volume of water is correct, microprocessor 82 sends a signal to activate the UV-C light tube 46, the hotplate 22, and the magnetic stirrer 24 to start heating the water to a specified temperature while also sterilizing the water and/or the apparatus 10 at the same time. When the specified temperature is reached, the microprocessor 82 sends a signal to activate/open a first gate 66 of a first cavity 64 so that a first pre-determined additive 15 is dispensed into the heated water. The first additive 15 is then stirred in the heated water for a specified period of time sufficient to dissolve the first additive 15 in the heated water. The water and first additive 15 mixture is then either heated or cooled to a second specified temperature and the microprocessor 82 sends a signal to activate/open a second gate 66 of a second cavity 64 so that a second pre-determined additive 15 is dispensed into the mixture. The second additive 15 is then stirred into the mixture for a second specified period of time sufficient to dissolve the second additive 15 in the mixture. The same cycle continues for a third and fourth additive 15 according to the formulation for the selected skin care product. After the last cycle, the mixture is cooled to a final specified temperature, the UV-C light 46 is deactivated, the door to the apparatus 10 is unlocked, and an on/off light will power off, indicating that the product is finished and ready to be dispensed for use.

While the embodiment of the disclosed apparatus and a method for preparing customized skin care products 10 has been set forth for purposes of illustration, the foregoing description should not be deemed a limitation of the invention. Accordingly, various modifications, adaptations and alternatives may occur to one skilled in the art without departing from the spirit of the disclosure and the scope of the claimed coverage.

1. An apparatus for preparing customized skin care products comprising:
a first portion including a hotplate for heating a liquid and an additive, a stirrer for mixing said liquid and said additive, a thermometer for determining the temperature of said liquid and said additive, and a scale for determining the weight of said liquid and said additive;
a second portion including a container defining a chamber for receiving said liquid and said additive;
a third portion including a dispenser defining a plurality of cavities for releasing said additive into said chamber; and
a control unit for receiving an input internal and external to said apparatus and outputting a signal to one or more of said first, second, and third portions,
wherein, when said control unit outputs said signal, one or more of said hotplate, said stirrer, said thermometer, said scale, or said dispenser are activated to prepare said customized skin care product.

2. The apparatus of claim 1, wherein said liquid and said additives are heated and mixed by said hotplate and said stirrer in said container.

3. The apparatus of claim 1, wherein each cavity includes a gate which opens upon receiving said signal from said control unit so that said additive is released into said container.

4. The apparatus of claim 1, wherein said additive comprises a plurality of pills housed within a pill cartridge.

5. The apparatus of claim 4, wherein when the dispenser is activated one or more of said pills are released into one or more of said cavities.

6. The apparatus of claim 1, wherein said control unit comprises a microprocessor connected to an RFID reader, said hotplate, said stirrer, said thermometer, said scale, and said dispenser.

7. The apparatus of claim 6, wherein said apparatus cooperates with an application to analyze skin of a user to recommend said additive to prepare said custom skin care product.

8. The apparatus of claim 1, wherein said control unit is connected to a UV-C light tube to sterilize an interior of said apparatus.

9. A method of preparing customized skin care products comprising:
determining the volume of a liquid within a container with a scale;
heating a liquid with a hotplate;
determining the temperature of the liquid with a thermometer;
releasing an additive stored in a cartridge into the liquid from a dispenser defining a plurality of cavities;
mixing said liquid and said additive with a stirrer;
outputting a signal from a control unit to one or more of said hotplate, said stirrer, said thermometer, said scale, or said dispenser;
wherein, outputting said signal from said control unit activates one or more of said hotplate, said stirrer, said thermometer, said scale, or said dispenser to prepare said customized skin care product.

10. The method of claim 9, further comprising a step of analyzing skin of a user to recommend said additive to prepare said custom skin care product.

11. The method of claim 9, further comprising a step of selecting one or more additives to prepare said custom skin care product based upon an analysis of skin of a user.

12. The method of claim 9, further comprising a step of weighing said liquid within said container.

13. The method of claim 9, further comprising a step of heating said liquid within said container.

14. The method of claim 9, further comprising a step of determining a temperature of said liquid within said container.

15. The method of claim 9, further comprising a step of dispensing a first additive from a first cavity of said plurality of cavities into said liquid within said container.

16. The method of claim 15, further comprising a step of stirring said liquid and said first additive within said container.

17. The method of claim 16, further comprising a step of dispensing a second additive from a second cavity of said plurality of cavities into said liquid within said container.

18. The method of claim 17, further comprising a step of stirring said liquid, said first additive, and said second additive within said container.

19. The method of claim 9, further comprising a step of dispensing said customized skin care product from said apparatus.

20. The method of claim 9, further comprising a step of sterilizing an interior of said apparatus with a UV-C light tube.
